(19) United States
(12) Patent Application Publication
(10) Pub. No.: US 2019/0141924 A1
(43) Pub. Date: May 16, 2019

(54) Relatively inexpensive process to turn green cells of Haematococcus pluvialis into astaxanthin accumulating red cysts

(71) Applicant: Arevik Minasyan, Glendale, CA (US)

(72) Inventor: Arevik Minasyan, Glendale, CA (US)

(21) Appl. No.: 15/947,809

(22) Filed: Apr. 7, 2018

Related U.S. Application Data
(60) Provisional application No. 62/584,922, filed on Nov. 13, 2017.

(51) Int. Cl. A01G 33/00; A01G 7/04; C12P 23/00

(52) U.S. Cl. CPC A01G 33/00; C12P 23/00; A01G 7/045

(57) ABSTRACT

This invention discloses an innovative budgetary process on how to turn green cells of Haematococcus pluvialis into red cysts that contain the astaxanthin of the highest rate. With this finding, green motile cells are converted into dormant red cysts via addition of resting cysts into the culture with green cells. Transformation of green cells of Haematococcus pluvialis into red dormant cells with this invented method can be achieved without need of high-intensity light. Light intensity can be reduced for up 15 times to approach the similar results within the same time-period. The least concentration of 1% of red cysts to green culture supports the economically most approved run. Shaking of the cultures of green cells with added red cysts stimulates the transformation process. Inoculation of higher concentration of red cysts into green culture of Haematococcus pluvialis shorten the period of greens conversion into a biomass, enriched with astaxanthin.

Nature has created a number of molecular mechanisms to accumulate specific secondary metabolites (i.e., pigments, vitamins, or lipids) to resist unfavorable environmental fluctuations (e.g. high light, salinity, nutrient stress, and high carbon/nitrogen ratio). One of such a protective system is a conversion of algal cells from green photosynthetic into dormant stage, when bioaccumulation of certain pigments take place in order to protect the algal cells from environmental unfavorable conditions.

One of such an example is Haematococcus pluvialis, a typical freshwater planktonic inhabitant, that synthesizes antioxidant pigment astaxanthin (red ketocarotenoid) of the highest value with up to 4% as free, and up to 94% and 2% as mono- and di-esterified (Holtin et al., 2009). The life cycles of Haematococcus pluvialis start with:—vegetative stage, when the cell is green and motile with two flagella;—green palmella, when the vegetative cell loses the flagella and enters the resting stage; and—the red cyst when the cell covers with thick cell-wall and accumulates the highest content of astaxanthin (Esra et al., 2007)

Since last decades astaxanthin (3,30-dihydroxy-b-b-carotene-4,40-dione) gained increased interest due to its applications in aquaculture, food, pharmaceutical, and nutraceutical industries (Lorenz and Cysewski, 2000), as well as a pigmentation inducer, and as an immune response enhancer and in anti-cancer treatment (Kobayashi et al., 1991).

Current invention describes a low-cost process about how to turn green cells of Haematococcus pluvialis into red cysts with the highest rate of astaxanthin accumulation. In order to achieve the final step of astaxanthin production by Haematococcus pluvialis, inoculation of resting cysts into the culture with green cells requires. Haematococcus pluvialis was grown in WC and BG-11 media. BG-11 media used in these analyses was modified. The concentration of dipotassium phosphate and sodium bicarbonate for stock solutions were reduced to 25M and 150M, respectively. Sodium carbonate was excluded from BG-11 media. Both media supported the finding similarly. Temperature was 22° C. Advantage of this invention is economically approved as transformation of green cells of Haematococcus pluvialis into red dormant cysts can be approached without need of high-intensity light. Light intensity can be reduced for up to 15 times from high intensity light of 600 lum ft⁻² to low intensity light of 40 lum ft⁻² to achieve the similar results within the same time-period. The least concentration of 1:100 with culture similar density of about 70.0*10³ L⁻¹ supports the economically most approved run. Shaking of the cultures of green cells with added red cysts stimulates the process of transformation into dormant stage. Inoculation of higher concentration of red cysts into green culture of Haematococcus pluvialis shorten the period of greens to convert into a biomass, enriched with astaxanthin. The tests of addition of 2%; 5%; 10%; 20%; and 50% of red cysts demonstrate the appearance of dormant cysts in period of 2-3 times shorter than if to stress green culture with high-light intensity. If to apply light intensity lower of 40 lum ft⁻² then the time, required to stress green cells of Haematococcus pluvialis and stimulate the process of astaxanthin accumulation, increases.

### DRAWINGS

FIG. 1. Illustration of transformation of green cells of Haematococcus pluvialis into red dormant cysts under high light intensity (light intensity of 600 lum ft⁻²).

FIG. 2. Illustration of transformation of green cells of Haematococcus pluvialis into dormant cells when red cysts are inoculated into green culture (ratio 1%) under low light intensity (light intensity of 40 lum ft⁻²).

FIG. 3. Illustration of transformation of green cells of Haematococcus pluvialis into dormant cells when red cysts are inoculated into green culture (ratio 2%) under low light intensity (light intensity of 40 lum ft⁻²).

FIG. 4. Illustration of transformation of green cells of Haematococcus pluvialis into dormant cells when red cysts are inoculated into green culture (ratio 10%) under low light intensity (light intensity of 40 lum ft⁻²).

FIG. 5. Illustration of transformation of green cells ofHaematococcus pluvialis into dormant cells when red cysts are inoculated into green culture (ratio 20%) under low light intensity (light intensity of 40 lum ft⁻²).

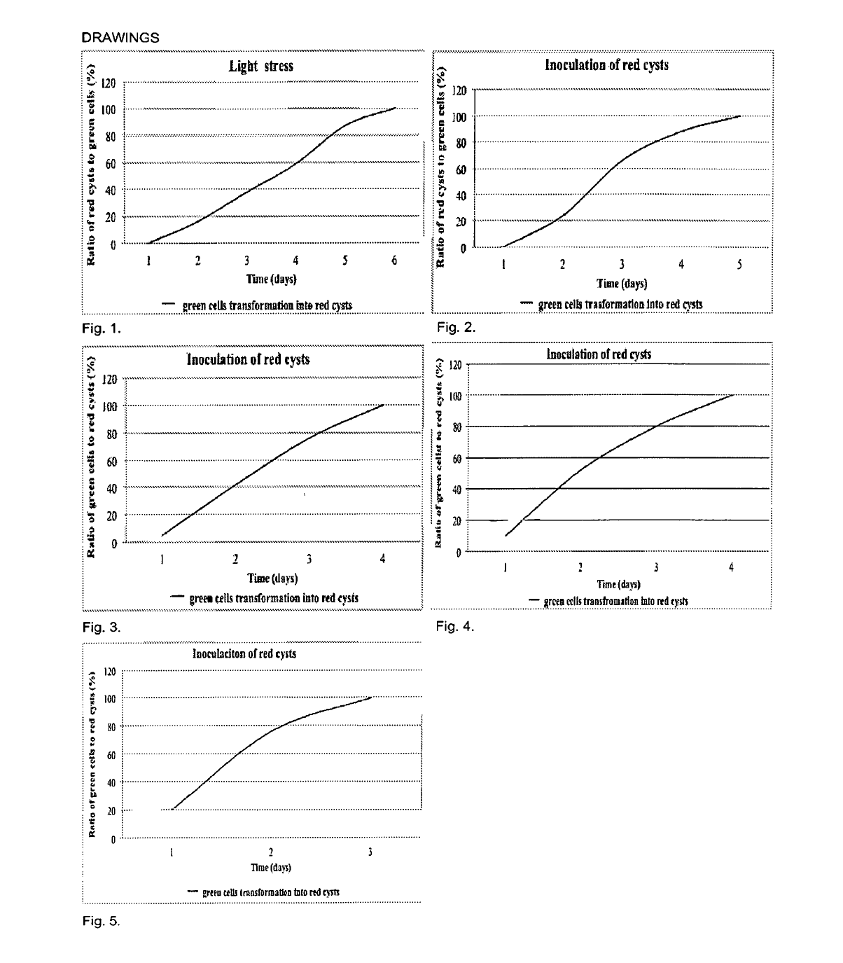

1. Addition of red dormant cysts to green culture of Haematococcus pluvialis demonstrates stimulating effect to turn green di-flagellates into resting cells even under low light conditions (from 600 lum ft⁻² to 40 lum ft⁻²) with the least ratio of 1:100 (dormant cysts : green cells of cells density 70.0*10³ L⁻¹) that requires similar period for green motile cells to convert into dormant cysts under high light stress.
Shaking of the green cultures after addition of red cells stimulates transmission of motile di-flagellates into resting stage.
Combination of high-intensity light (300-600 lum ft⁻²) with inoculation of dormant cells in green culture reduces time-frame up to 2 times, and if to synchronize these together with shaking, it reduces period of greens transformation into dormant stage in up to 3 times.
